(19) United States
(12) Patent Application Publication
(10) Pub. No.: US 2026/0272921 A1
(43) Pub. Date: Sep. 17, 2026

(54) RAS AND PRMT5 INHIBITORS COMBINATION THERAPY

(71) Applicant: Erasca, Inc., San Diego, CA (US)

(72) Inventors: Robert Field SHOEMAKER, San Diego, CA (US); Alexei BROOUN, San Diego, CA (US); Jae Hyun BAE, San Diego, CA (US)

(21) Appl. No.: 19/664,682

(22) Filed: Apr. 30, 2026

Related U.S. Application Data
(63) Continuation of application No. PCT/US2025/042837, filed on Aug. 20, 2025.
(60) Provisional application No. 63/685,384, filed on Aug. 21, 2024.

(51) Int. Cl. A61K 31/504; A61K 31/4545; A61P 35/00

(52) U.S. Cl. CPC A61K 31/504; A61K 31/4545; A61P 35/00

(57) ABSTRACT

The present disclosure relates generally to the use of a Pan-RAS inhibitor in combination with a PRMT5 inhibitor for treating cancer, specifically solid tumors.

### CROSS-REFERENCE

This application is a continuation of International Application No. PCT/US2025/042837, filed Aug. 20, 2025, which claims priority to U.S. Provisional Application Ser. No. 63/685,384, filed Aug. 21, 2024; which are hereby incorporated by reference in their entirety.

### BACKGROUND

The RAS-RAF-MEK-ERK signal transduction pathway, which is sometimes denoted as the mitogen-activated protein kinase (MAPK) pathway is thought to play a central role in regulating a number of fundamental cellular processes including one or more of cell proliferation, survival, adhesion, cycle progression, migration, differentiation, metabolism, and transcription. The activation of the MAPK pathway has been reported in numerous tumor types including lung, colon, pancreatic, renal, and ovarian cancers. Accordingly, substances that could reduce activation could be of interest for possible treatments.

### SUMMARY

The ERK1/2 (RAS, RAF, MEK, ERK) and PI3K (PI3K, AKT, mTOR, PTEN) pathways are the chief signaling pathways for cellular proliferation, survival, and differentiation. Overactivation and hyperphosphorylation of the ERK1/2 & PI3K pathways is frequently observed in cancer and is associated with poor patient prognosis. While it is well known that genetic alterations lead to the dysregulation of the ERK1/2 & PI3K pathways, increasing evidence showcase that epigenetic alterations also play a major role in the regulation of the ERK1/2 & PI3K pathways. Protein Arginine Methyltransferase 5 (PRMT5) is a posttranslational modifier for multiple cellular processes, which is currently being tested as a therapeutic target for cancer. PRMT5 has been shown to be overexpressed in many types of cancers, as well as negatively correlated with patient survival. Numerous studies are indicating that as a posttranslational modifier, PRMT5 is extensively involved in regulating the ERK1/2 & PI3K pathways.

The present embodiments disclosed herein generally relate to compositions and methods related to combination therapies to treat cancer utilizing either a KRAS selective inhibitor or a Pan-RAS inhibitor in conjunction with a PRMT5 inhibitor while providing an unexpected degree of synergy.

Disclosed herein is a method of treating cancer in a subject in need thereof, the method comprising: administering to the subject in need thereof a therapeutically effective amount of:
(i) Compound 2:

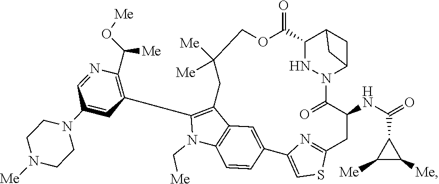

or a pharmaceutically acceptable salt thereof; and
(ii) TNG-462;
wherein the cancer is an MTAP-deleted cancer (MTAP^{del}).

In some embodiments, the cancer is a RAS-dependent cancer. In some embodiments, the cancer comprises a RAS mutation. In some embodiments, the RAS mutation is a KRAS, a NRAS, and/or a HRAS mutation. In some embodiments, the RAS mutation is a mutation at the G12, G13, and/or Q61 position of the RAS protein. In some embodiments, the RAS mutation is a G12C mutation of the RAS protein. In some embodiments, the RAS mutation is a G12D mutation of the RAS protein. In some embodiments, the RAS mutation is a G12R mutation of the RAS protein. In some embodiments, the RAS mutation is a G12V mutation of the RAS protein. In some embodiments, the cancer is a solid tumor. The method of claim 1, wherein the cancer is breast cancer, gastrointestinal cancer, head and neck cancer, lung cancer, liver cancer, melanoma, ovarian cancer, pancreatic cancer, salivary gland tumor, thyroid cancer, or uterine cancer. In some embodiments, the lung cancer is non-small cell lung cancer (NSCLC). In some embodiments, the pancreatic cancer is pancreatic ductal adenocarcinoma (PDAC). In some embodiments, the gastrointestinal cancer is anal cancer, appendiceal cancer, bile duct cancer, cholangiocarcinoma cancer, colon cancer, colorectal cancer (CRC), gallbladder cancer, rectal cancer, small intestine cancer, or stomach cancer (gastric cancer). In some embodiments, the gastrointestinal cancer is colorectal cancer (CRC). In some embodiments, the amount of Compound 2, or a pharmaceutically acceptable salt thereof, administered is between 1 mg and 100 mg per day. In some embodiments, the amount of Compound 2, or a pharmaceutically acceptable salt thereof, administered is between 1 mg and 80 mg per day. In some embodiments, the amount of Compound 2, or a pharmaceutically acceptable salt thereof, administered is between 1 mg and 60 mg per day. In some embodiments, the amount of Compound 2, or a pharmaceutically acceptable salt thereof, administered is between 1 mg and 40 mg per day. In some embodiments, the amount of Compound 2, or a pharmaceutically acceptable salt thereof, administered is between 1 mg and 20 mg per day.

### BRIEF DESCRIPTION OF THE DRAWINGS

FIG. 1 illustrates the HSA synergy and antagonism of Compound 2 and TNG-462 in LU99 (KRAS^{G12C}, MTAP^{del}, NSCLC) measured with the CellTiter-Glo® Luminescent 3D Cell Viability assay.

FIG. 2 illustrates the HSA synergy and antagonism of Compound 2 and MRTX-1719 in LU99 (KRAS^{G12C}, MTAP^{del}, NSCLC) measured with the CellTiter-Glo® Luminescent 3D Cell Viability assay.

FIG. 3 illustrates the HSA synergy and antagonism of Compound 2 and TNG-462 in PSN-1 (KRAS^{G12R}, MTAP^{del}, NSCLC) measured with the CellTiter-Glo® Luminescent 3D Cell Viability assay.

FIG. 4 illustrates the HSA synergy and antagonism of Compound 2 and MRTX-1719 in PSN-1 (KRAS^{G12R}, MTAP^{del}, NSCLC) measured with the CellTiter-Glo® Luminescent 3D Cell Viability assay.

FIG. 5 illustrates the HSA synergy and antagonism of Compound 2 and TNG-462 in HuP-T3 (KRAS^{G12R}, MTAP^{del}, PDAC) measured with the CellTiter-Glo® Luminescent Cell Viability Assay.

FIG. 6 illustrates the HSA synergy and antagonism of Compound 2 and MRTX-1719 in HuP-T3 (KRAS^{G12R}, MTAP^{del}, PDAC) measured with the CellTiter-Glo® Luminescent Cell Viability Assay.

FIG. 7 illustrates the HSA synergy and antagonism of Compound 2 and TNG-462 in SU86.86 (KRASG12D, MTAP^{del}, PDAC) measured with the CellTiter-Glo® Luminescent 3D Cell Viability assay.

FIG. 8 illustrates the HSA synergy and antagonism of Compound 2 and MRTX-1719 in SU86.86 (KRAS^{G12D}, MTAP^{del}, PDAC) measured with the CellTiter-Glo® Luminescent 3D Cell Viability assay.

FIG. 9 illustrates the HSA synergy and antagonism of Compound 2 and MRTX-1719 in Capan-1 (KRAS^{G12V}, MTAP^{del}, PDAC) measured with the CellTiter-Glo® Luminescent Cell Viability Assay.

FIG. 10 illustrates the HSA synergy and antagonism of Compound 2 and TNG-462 in Capan-1 (KRAS^{G12V}, MTAP^{del}, PDAC) measured with the CellTiter-Glo® Luminescent Cell Viability Assay.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION

As used herein and in the appended claims, the singular forms “a,” “an,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth. When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range, in some instances, will vary between 1% and 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of” the described features.

As used in the specification and appended claims, unless specified to the contrary, the following terms have the meaning indicated below.

As used herein, the term “therapeutic” means an agent utilized to treat, combat, ameliorate, prevent, or improve an unwanted condition or disease of a patient. In some embodiments, a therapeutic agent such as a compound 1 is directed to the treatment and/or the amelioration of cancers.

“Administering,” when used in conjunction with a therapeutic means to administer a therapeutic systemically or locally, as directly into or onto a target tissue, or to administer a therapeutic to a patient whereby the therapeutic positively impacts the tissue to which it is targeted. Thus, as used herein, the term “administering,” when used in conjunction with a composition described herein, can include, but is not limited to, providing a composition into or onto the target tissue; providing a composition systemically to a patient by, e.g., oral administration whereby the therapeutic reaches the target tissue or cells. “Administering” a composition may be accomplished by injection, topical administration, and oral administration or by other methods alone or in combination with other known techniques.

The term “animal” as used herein includes, but is not limited to, humans and non-human vertebrates such as wild, domestic and farm animals. As used herein, the terms “patient,” “subject” and “individual” are intended to include living organisms in which certain conditions as described herein can occur. Examples include humans, monkeys, cows, sheep, goats, dogs, cats, mice, rats, and transgenic species thereof. In a preferred embodiment, the patient is a primate. In certain embodiments, the primate or subject is a human. In certain instances, the human is an adult. In certain instances, the human is child. In further instances, the human is under the age of 12 years. In certain instances, the human is elderly. In other instances, the human is 60 years of age or older. Other examples of subjects include experimental animals such as mice, rats, dogs, cats, goats, sheep, pigs, and cows. The experimental animal can be an animal model for a disorder, e.g., a transgenic mouse with hypertensive pathology.

By “pharmaceutically acceptable,” it is meant the carrier, diluent or excipient must be compatible with the other ingredients of the formulation and not deleterious to the recipient thereof.

The term “pharmaceutical composition” shall mean a composition comprising at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

A “therapeutically effective amount” or “effective amount” as used herein refers to the amount of active compound or pharmaceutical agent that elicits a biological or medicinal response in a tissue, system, animal, individual or human that is being sought by a researcher, veterinarian, medical doctor or other clinician, which includes one or more of the following: (1) preventing the disease; for example, preventing a disease, condition or disorder in an individual that may be predisposed to the disease, condition or disorder but does not yet experience or display the pathology or symptomatology of the disease, (2) inhibiting the disease; for example, inhibiting a disease, condition or disorder in an individual that is experiencing or displaying the pathology or symptomatology of the disease, condition or disorder (i.e., arresting further development of the pathology and/or symptomatology), and (3) ameliorating the disease; for example, ameliorating a disease, condition or disorder in an individual that is experiencing or displaying the pathology or symptomatology of the disease, condition or disorder (i.e., reversing the pathology and/or symptomatology).

The terms “treat,” “treated,” “treatment,” or “treating” as used herein refers to therapeutic treatment, wherein the object is to prevent or slow (lessen) an undesired physiological condition, disorder, or disease, or to obtain beneficial or desired clinical results. For the purposes described herein, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment.

### Compound 1

Disclosed herein is compound 1:

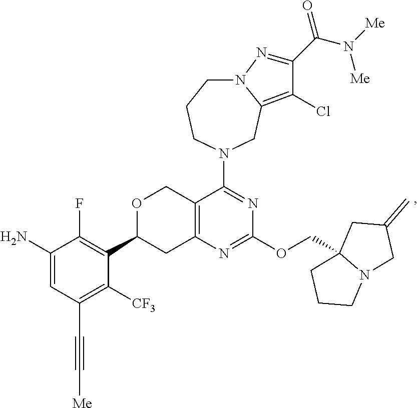 [SMILES: C=C1CN2CCC[C@@]2(COc2nc3c(c(N4CCCn5nc(C(=O)N(C)C)c(Cl)c5C4)n2)CO[C@H](c2c(F)c(N)cc(C#CC)c2C(F)(F)F)C3)C1]

or a pharmaceutically acceptable salt thereof. In some embodiments, Compound 1 is not a salt. In some embodiments, the salt of Compound 1 is the dihydrochloride salt.

In some embodiments, Compound 1 is a potent and selective inhibitor of KRAS that has the potential to provide an improved therapeutic window relative to RAS inhibitors and prevent KRAS wildtype-mediated resistance relative to mutant-selective approaches.

### Compound 2

Disclosed herein is the pan-RAS molecular glue Compound 2:

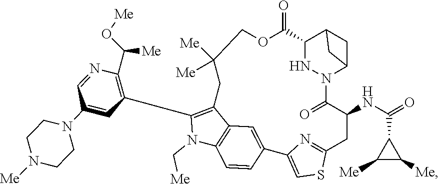

or a pharmaceutically acceptable salt thereof.

In some embodiments, Compound 2 is not a salt.

### PRMT5 Inhibitor

PRMT5 is overexpressed in solid tumors and hematological malignancies, and is associated with cancer initiation and progression.

In some embodiments, the PRMT5 inhibitor is AMG 193, AZD3470, GSK3326595, JNJ-64619178, MRTX-1719, PF-06939999, PRT543, PRT811, SCR-6920, TNG-462, or TNG908.

In some embodiments, the PRMT5 inhibitor is MRTX-1719 or TNG-462. In some embodiments, the PRMT5 inhibitor is MRTX-1719. In some embodiments, the PRMT5 inhibitor is TNG-462.

In some embodiments, the PRMT5 inhibitor is an inhibitor disclosed in one of the following PCT applications: WO2011079236, WO2014100764, WO2014100764, WO2014100716, WO2014100695, WO2014100734, WO2014100730, WO2014100719, WO2014100719, WO2014145214, WO2014145214, WO2015200677, WO2015200677, WO2015200677, WO2015200680, WO2015200680, WO2015200680, WO2016022605, WO2016034671, WO2016034675, WO2016034673, WO2016038550, WO2016089883, WO2016145150, WO2016145150, WO2017032840, WO2017153518, WO2017153515, WO2017153513, WO2017153186, WO2017218802, WO2018065365, WO2018075601, WO2018081451, WO2018085818, WO2018085833, WO2018085833, WO2018152501, WO2018152548, WO2018152548, WO2018154104, WO2018160824, WO2018160855, WO2018161922, WO2018167276, WO2018167269, WO2019002074, WO2019032859, WO2019084470, WO2019094311, WO2019094312, WO2019102494, WO2019110734, WO2019112719, WO2020243178, WO2019116302, WO2019165189, WO2019173804, WO2019178368, WO2019180628, WO2019180631, WO2020033282, WO2020033285, WO2020033288, WO2020033284, WO2020094712, WO2020168125, WO2020198323, WO2020205867, WO2020205660, WO2020217070, WO2020245365, WO2020250123, WO2020259478, WO2021050915, WO2021055797, WO2021055797, WO2021066578, WO2021068953, WO2021080955, WO2021079302, WO2021080359, WO2021088992, WO2021111322, WO2021124096, WO2021126729, WO2021126731, WO2021126999, WO2021126728, WO2021126732, WO2021138578, WO2021140427, WO2021163344, WO2021202480, WO2021207052, WO2021225963, WO2021225963, WO2021244542, WO2021254493, WO2022002142, WO2022026892, WO2022032144, WO2022048631, WO2022115377, WO2022125735, WO2022132914, WO2022153161, WO2022169948, WO2022192745, WO2022206964, WO2022216645, WO2022216648, WO2022237858, WO2022258986, WO2023278564, WO2023001133, WO2023059795, WO2023081367, WO2023081367, WO2023094839, WO2023098439, WO2023114507, WO2023125540, WO2023125947, WO2023131305, WO2023143210, WO2023175319, WO2023174250, WO2023196545, WO2023202626, WO2023207556, WO2023223099, WO2023223099, WO2023245037, WO2024002376, WO2024002377, WO2024008113, WO2024008176, WO2024012308, WO2024022433, WO2024022186, WO2024021957, WO2024027703, WO2024027370, WO2024032572, WO2024067433, WO2024067445, WO2024039864, WO2024037608, WO2024037607, WO2024038004, WO2024049948, WO2024051816, WO2024074611, WO2024101965, WO2024104244, WO2024153128, WO2024118897, WO2024118943, WO2024118943, WO2024131901, WO2024131918, WO2024137852, WO2024137778, WO2024140923, WO2024152973, WO2024153230, or WO2024160058.

In some embodiments, the PRMT5 inhibitor is an inhibitor disclosed in WO2023125540.

In some embodiments, the PRMT5 inhibitor is an inhibitor disclosed in WO2022026892.

In some embodiments, the PRMT5 inhibitor is a compound of Formula (I):

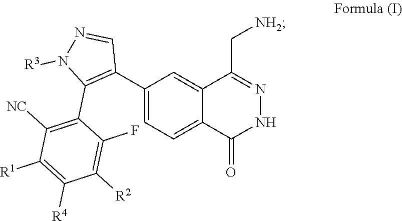

wherein:
R¹ is C₂₋₅alkenyl, C₂₋₅alkynyl, —OC₃₋₅cycloalkyl, —OC₂₋₃alkynyl, or C₃₋₅cycloalkyl, wherein the alkenyl, alkynyl, and cycloalkyl are independently optionally substituted by 1, 2, or 3 R^{a};
R² is halogen or C₂₋₃alkynyl optionally substituted by 1, 2, or 3 halogens;
R³ is CH₃ or CD₃;
R⁴ is hydrogen or halogen; and
each Rₐ is independently halogen or C₁₋₃alkyl optionally substituted by 1, 2, or 3 halogens;
or a pharmaceutically acceptable salt thereof.

In some embodiments, the PRMT5 inhibitor is

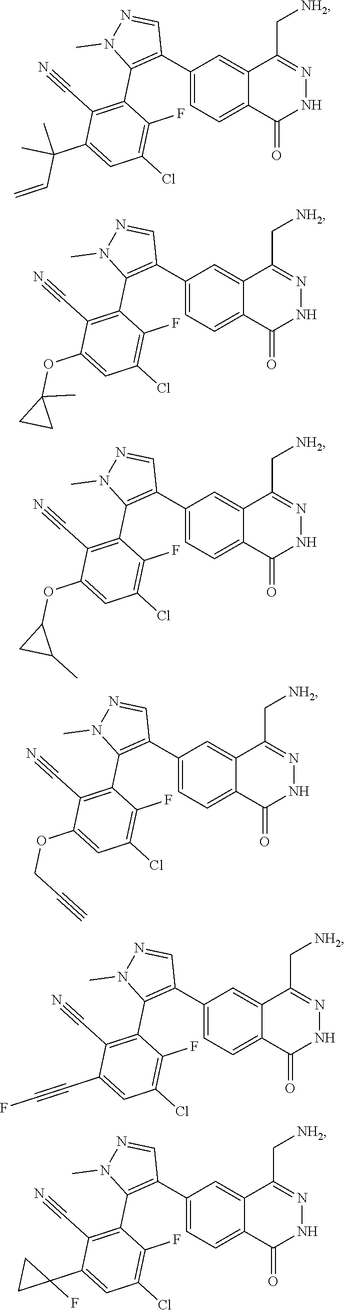

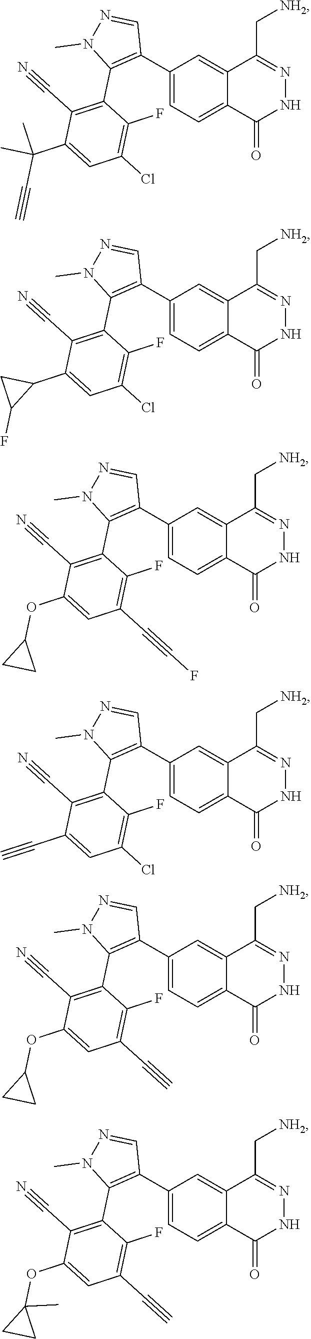

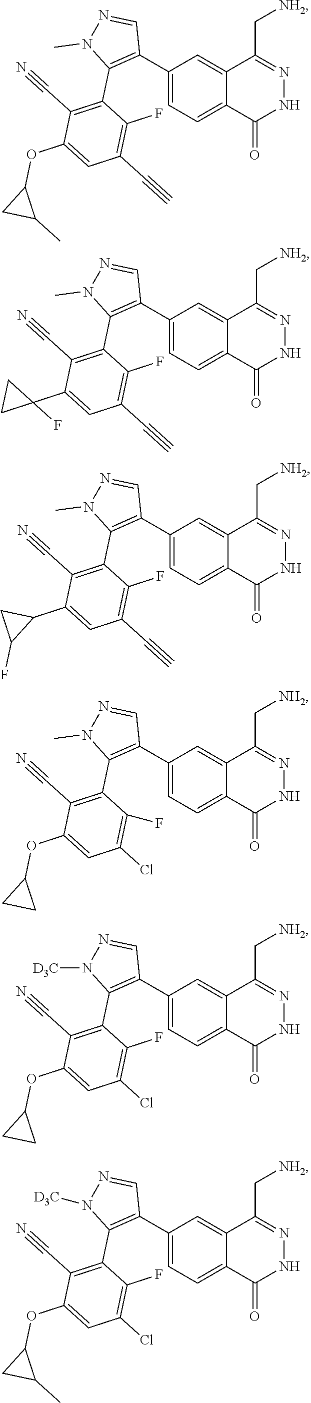 [SMILES: C#Cc1cc(OC2CC2C)c(C#N)c(-c2c(-c3ccc4c(=O)[nH]nc(CN)c4c3)cnn2C)c1F ; C#Cc1cc(C2(F)CC2)c(C#N)c(-c2c(-c3ccc4c(=O)[nH]nc(CN)c4c3)cnn2C)c1F ; C#Cc1cc(C2CC2F)c(C#N)c(-c2c(-c3ccc4c(=O)[nH]nc(CN)c4c3)cnn2C)c1F ; Cn1ncc(-c2ccc3c(=O)[nH]nc(CN)c3c2)c1-c1c(F)c(Cl)cc(OC2CC2)c1C#N ; [2H]C([2H])([2H])n1ncc(-c2ccc3c(=O)[nH]nc(CN)c3c2)c1-c1c(F)c(Cl)cc(OC2CC2)c1C#N ; [2H]C([2H])([2H])n1ncc(-c2ccc3c(=O)[nH]nc(CN)c3c2)c1-c1c(F)c(Cl)cc(OC2CC2C)c1C#N]

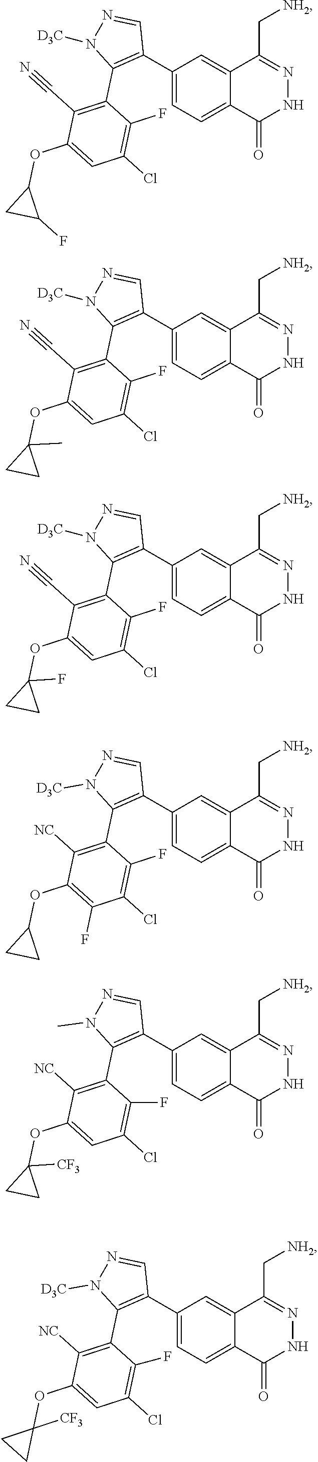

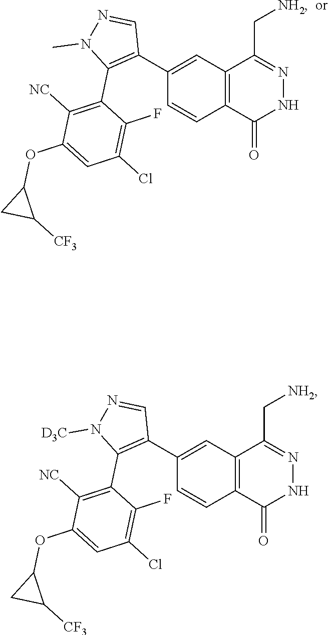

or a pharmaceutically acceptable salt thereof.

In some embodiments, the PRMT5 inhibitor is

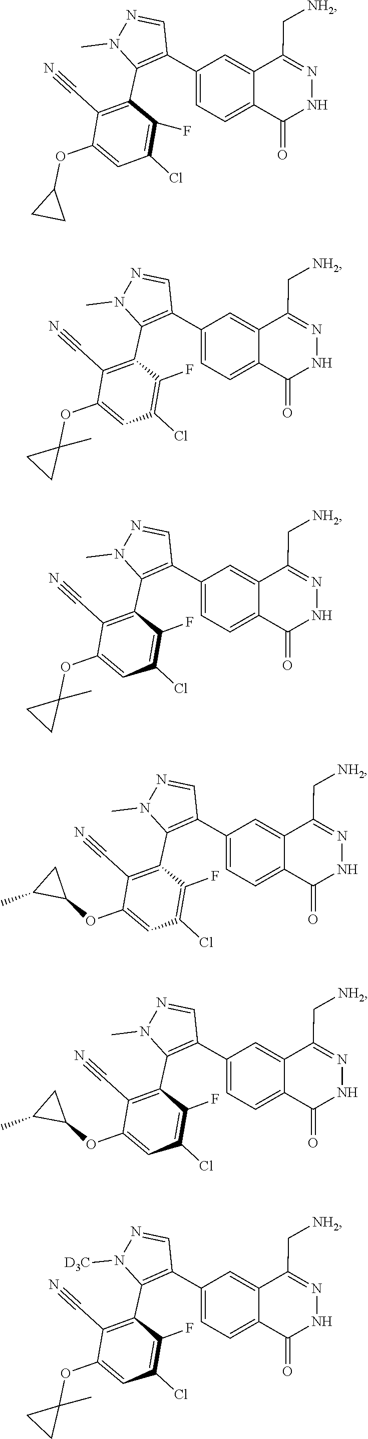

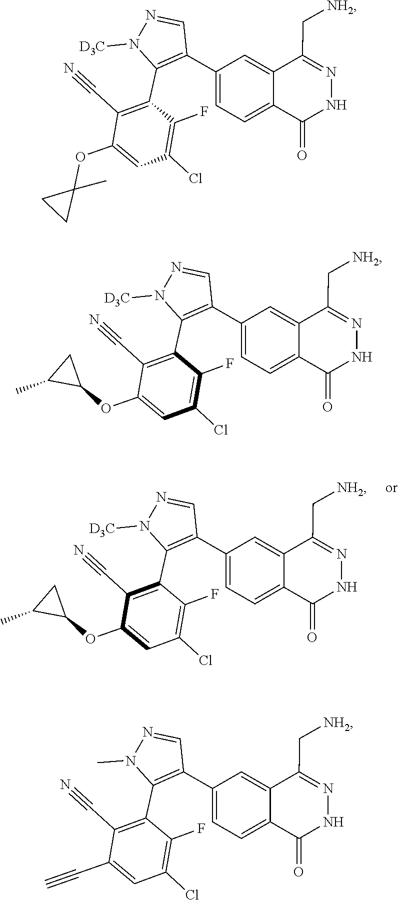

or a pharmaceutically acceptable salt thereof.

In some embodiments, the PRMT5 inhibitor is

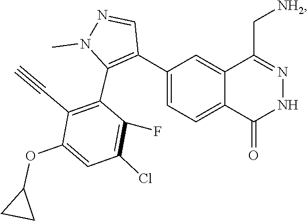 [SMILES: C#Cc1c(OC2CC2)cc(Cl)c(F)c1-c1c(-c2ccc3c(=O)[nH]nc(CN)c3c2)cnn1C]

or a pharmaceutically acceptable salt thereof.

In some embodiments, the PRMT5 inhibitor is a compound of Formula (II):

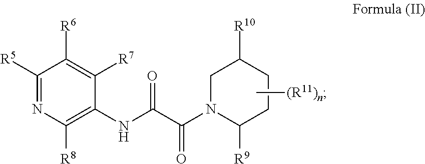

wherein:
R⁵ is independently selected from the group consisting of H, —C₁-C₆ alkyl, —OR^{a1} and —N(R^{a1})₂;
each R^{a1} is independently selected from the group consisting of H, —C₁-C₆ alkyl and —C₁-C₆ haloalkyl;
R⁶ is independently selected from the group consisting of halo, —C₁-C₆ alkyl, —C₁-C₆ haloalkyl, —C₃-C₉ cycloalkyl, 3-10 membered heterocyclyl, —OR^{a2} and —C(═O)N(R^{a2})₂;
R^{a2} is independently selected from the group consisting of H and —C₁-C₆ alkyl;
R⁷ is H;
R⁸ is H;
R⁹ is

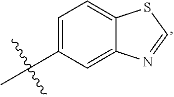 [SMILES: *c1ccc2scnc2c1]

which is unsubstituted or substituted with 1 instance of R^{a9};
R¹⁰ is selected from the group consisting of —H and —CH₃;
each R¹¹ is independently selected from the group consisting of halo, —C₁-C₆ alkyl and —OR^{a11};
each R^{a11} is independently selected from the group consisting of H, —CF₃ and —C₁-C₆ alkyl;
each R^{a9} is independently selected from the group consisting of -D, —C₁-C₆ alkyl, —C₁-C₆ heteroalkyl, —C₁-C₆ haloalkyl, —C₃-C₉ cycloalkyl, 3-10 membered heterocyclyl, 5-10 membered heteroaryl, heterocyclylalkyl, and —N(R^{b10})₂, wherein each alkyl, cycloalkyl, heteroalkyl, heterocyclyl, heteroaryl and heterocyclylalkyl of R¹⁰ is substituted with 0, 1, 2, 3, 4, or 5 instances of -Me, -Et, -ⁱPr, cyclopropyl, oxetan-3-yl, —OH, ═O, —F, —OMe, —CH₂CH₂F, —CH₂CHF₂, —CH₂CH₂CF₃, —C(═O)Me, —N(Me)₂, —CH₂N(CH₃)₂ or a combination thereof;
each R^{b10} is independently selected from the group consisting of H and —C₁-C₆ alkyl; and
n is 0, 1 or 2.

In some embodiments, the PRMT5 inhibitor is:

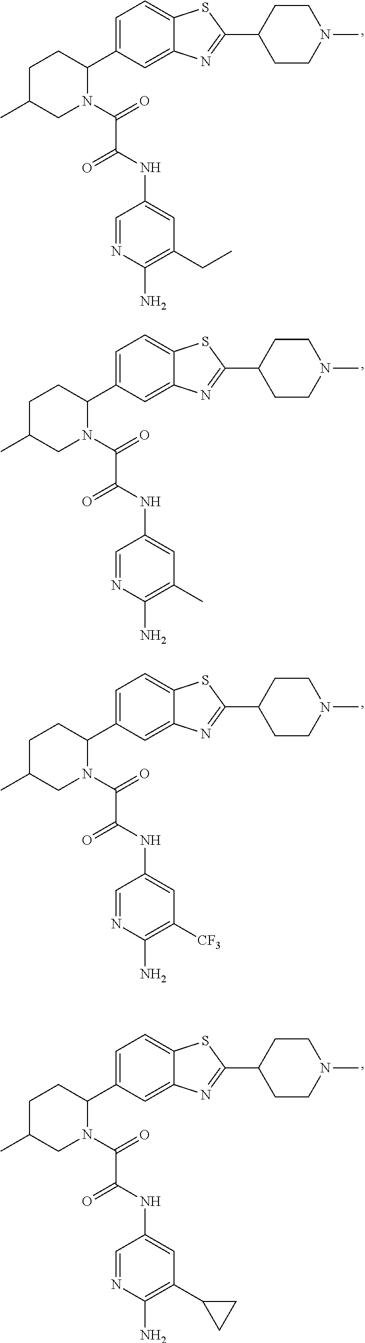

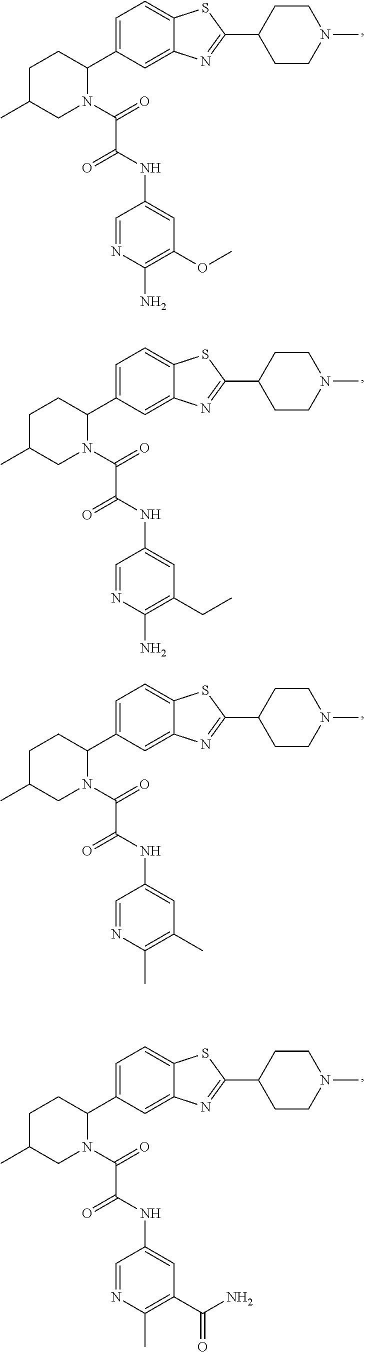

or a pharmaceutically acceptable salt thereof.

In some embodiments, the PRMT5 inhibitor is:

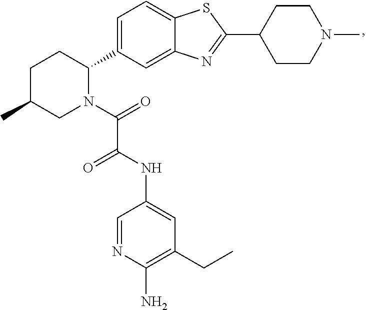

or a pharmaceutically acceptable salt thereof.

### Combinations

Disclosed herein is a method of treating cancer in a subject in need thereof, the method comprising: administering to the subject in need thereof a therapeutically effective amount of
(i) Compound 1:

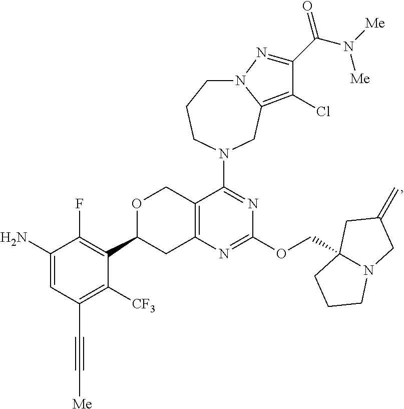 [SMILES: C=C1CN2CCC[C@@]2(COc2nc3c(c(N4CCCn5nc(C(=O)N(C)C)c(Cl)c5C4)n2)CO[C@H](c2c(F)c(N)cc(C#CC)c2C(F)(F)F)C3)C1]

or a pharmaceutically acceptable salt thereof; and
(ii) a PRMT5 inhibitor.

Also disclosed herein is a method of treating cancer in a subject in need thereof, the method comprising: administering to the subject in need thereof a therapeutically effective amount of
(i) Compound 2:

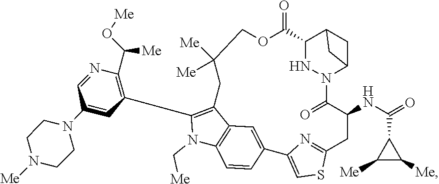

or a pharmaceutically acceptable salt thereof; and
(ii) a PRMT5 inhibitor.

Also disclosed herein is a method of treating cancer in a subject in need thereof, the method comprising: administering to the subject in need thereof a therapeutically effective amount of
(i) Compound 1:

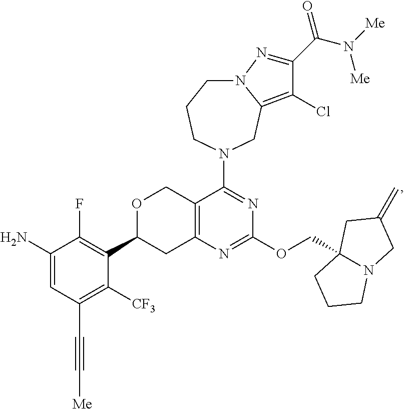 [SMILES: C=C1CN2CCC[C@@]2(COc2nc3c(c(N4CCCn5nc(C(=O)N(C)C)c(Cl)c5C4)n2)CO[C@H](c2c(F)c(N)cc(C#CC)c2C(F)(F)F)C3)C1]

or a pharmaceutically acceptable salt thereof;
(ii) Compound 2:

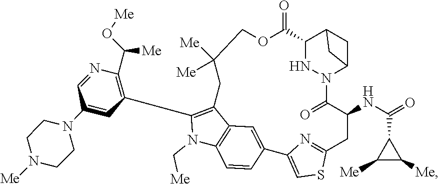

or a pharmaceutically acceptable salt thereof; and
(iii) a PRMT5 inhibitor.

Disclosed herein is a compound that is Compound 1 or its pharmaceutically acceptable salt:

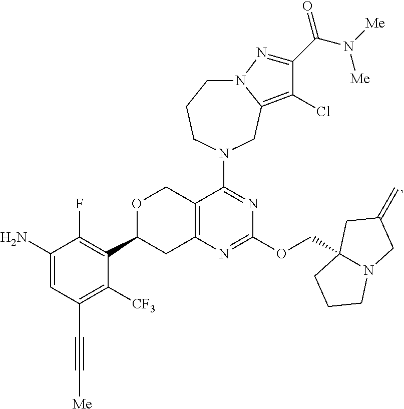 [SMILES: C=C1CN2CCC[C@@]2(COc2nc3c(c(N4CCCn5nc(C(=O)N(C)C)c(Cl)c5C4)n2)CO[C@H](c2c(F)c(N)cc(C#CC)c2C(F)(F)F)C3)C1]

for use in a method of treating a subject having cancer,
the method comprising administering to said subject a therapeutically effective amount of said Compound 1 or salt thereof in combination with a PRMT5 inhibitor.

Also disclosed herein is a compound that is Compound 2 or its pharmaceutically acceptable salt:

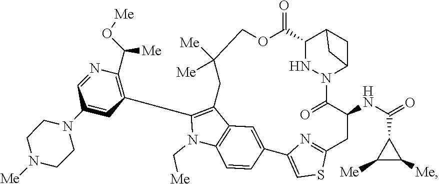

for use in a method of treating a subject having cancer,
the method comprising administering to said subject a therapeutically effective amount of said Compound 1 or salt thereof in combination with a PRMT5 inhibitor.

Also disclosed herein is a compound that is Compound 1 or its pharmaceutically acceptable salt:

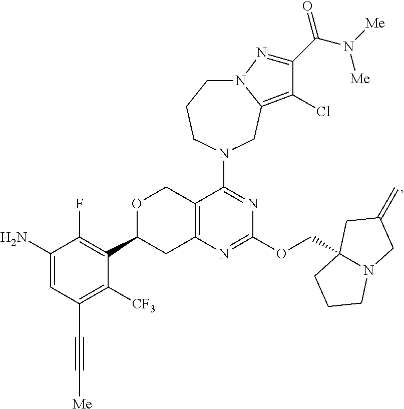 [SMILES: C=C1CN2CCC[C@@]2(COc2nc3c(c(N4CCCn5nc(C(=O)N(C)C)c(Cl)c5C4)n2)CO[C@H](c2c(F)c(N)cc(C#CC)c2C(F)(F)F)C3)C1]

for use in a method of treating a subject having cancer,
the method comprising administering to said subject a therapeutically effective amount of said Compound 1 or salt thereof in combination with Compound 2:

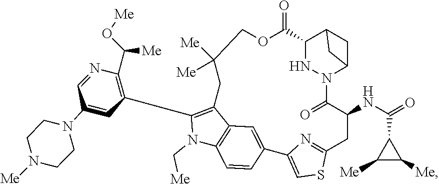

or its pharmaceutically acceptable salt and in combination with a PRMT5 inhibitor.

Also disclosed herein is a compound that is Compound 2 or its pharmaceutically acceptable salt:

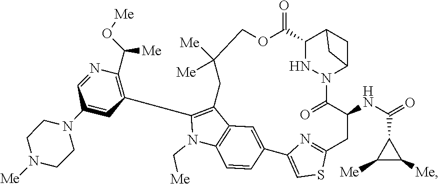

for use in a method of treating a subject having cancer,
the method comprising administering to said subject a therapeutically effective amount of said Compound 2 or salt thereof in combination with Compound 1:

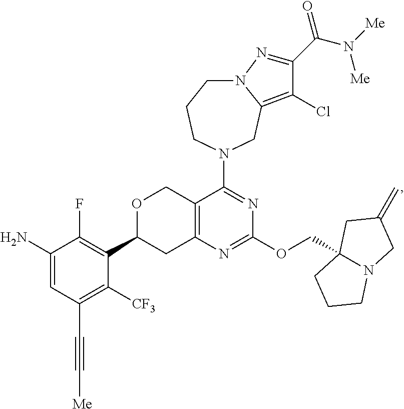 [SMILES: C=C1CN2CCC[C@@]2(COc2nc3c(c(N4CCCn5nc(C(=O)N(C)C)c(Cl)c5C4)n2)CO[C@H](c2c(F)c(N)cc(C#CC)c2C(F)(F)F)C3)C1]

or its pharmaceutically acceptable salt and in combination with PRMT5 inhibitor.

### Cancers

Disclosed herein are methods of treating cancer using a combination disclosed herein.

“Cancer” refers to all types of cancer, neoplasm or malignant tumors found in mammals (e.g. humans), including, without limitation, leukemias, lymphomas, myelomas, carcinomas, and sarcomas. Exemplary cancers that may be treated with a compound or method provided herein include brain cancer, glioma, glioblastoma, neuroblastoma, prostate cancer, colorectal cancer, pancreatic cancer (such as pancreatic adenocarcinoma, PDAC), medulloblastoma, melanoma, cervical cancer, gastric cancer, ovarian cancer, lung cancer, cancer of the head, Hodgkin's Disease, and Non-Hodgkin's Lymphomas. Exemplary cancers that may be treated with a compound or method provided herein include cancer of the blood, thyroid, endocrine system, brain, breast, cervix, colon, head & neck, liver, kidney, lung, ovary, pancreas, rectum, stomach, and uterus. Additional examples include, thyroid carcinoma, cholangiocarcinoma, pancreatic adenocarcinoma, skin cutaneous melanoma, colon adenocarcinoma, rectum adenocarcinoma, stomach adenocarcinoma, esophageal carcinoma, head and neck squamous cell carcinoma, breast invasive carcinoma, lung adenocarcinoma, lung squamous cell carcinoma, non-small cell lung carcinoma, mesothelioma, multiple myeloma, neuroblastoma, glioma, glioblastoma multiforme, ovarian cancer, rhabdomyosarcoma, primary thrombocytosis, primary macroglobulinemia, primary brain tumors, malignant pancreatic insulanoma, malignant carcinoid, urinary bladder cancer, premalignant skin lesions, testicular cancer, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

In some embodiments, the cancer harbors at least one of a KRAS, NRAS, or HRAS mutations.

In some embodiments, the cancer is a KRAS-driven cancer, HRAS-driven cancer, or a NRAS-driven cancer.

In some embodiments, the cancer has at least one mutation in RAS.

In some embodiments, the RAS mutation is a mutation at the G12, G13, and/or Q61 position of the RAS protein.

In some embodiments, the cancer has a G12C RAS mutation. In some embodiments, the cancer has a G12D RAS mutation. In some embodiments, the cancer has a G12R RAS mutation. In some embodiments, the cancer has a G12S RAS mutation. In some embodiments, the cancer has a G12V RAS mutation. In some embodiments, the cancer has G12W RAS mutation. In some embodiments, the cancer has a G13D RAS mutation. In some embodiments, the cancer has a H95D RAS mutation. In some embodiments, the cancer has a H95Q RAS mutation. In some embodiments, the cancer has a H95R RAS mutation. In some embodiments, the cancer has a Q16H RAS mutation. In some embodiments, the cancer has a Q61H RAS mutation. In some embodiments, the cancer has a Q16K RAS mutation. In some embodiments, the cancer has a Q61R RAS mutation. In some embodiments, the cancer has a R68S RAS mutation.

In some embodiments, the cancer is the cancer is a liquid tumor. In some embodiments, the cancer is the liquid tumor is leukemia. In some embodiments, the cancer is the leukemia is acute myeloid leukemia (AML).

In some embodiments, the cancer is a solid tumor. In some embodiments, the solid tumor is an advanced or a metastatic solid tumor.

In some embodiments, the cancer is breast cancer, esophageal cancer, gastrointestinal cancer, head and neck cancer, lung cancer, liver cancer, melanoma, ovarian cancer, pancreatic cancer, salivary gland tumor, thyroid cancer, or uterine cancer.

In some embodiments, the lung cancer is non-small cell lung cancer (NSCLC).

In some embodiments, the pancreatic cancer is pancreatic ductal adenocarcinoma (PDAC).

In some embodiments, the gastrointestinal cancer is anal cancer, appendiceal cancer, bile duct cancer, cholangiocarcinoma cancer, colon cancer, colorectal cancer (CRC), gallbladder cancer, rectal cancer, small intestine cancer, or stomach cancer (gastric cancer).

### Dosing

In one aspect, the compositions described herein are used for the treatment of diseases and conditions described herein. In addition, a method for treating any of the diseases or conditions described herein in a subject in need of such treatment, involves administration of compositions in therapeutically effective amounts to said subject.

Dosages of compositions described herein can be determined by any suitable method. Maximum tolerated doses (MTD) and maximum response doses (MRD) for compound 1, or a pharmaceutically acceptable salt thereof can be determined via established animal and human experimental protocols as well as in the examples described herein. For example, toxicity and therapeutic efficacy of compound 1, or a pharmaceutically acceptable salt thereof, can be determined by standard pharmaceutical procedures in cell cultures or experimental animals, including, but not limited to, for determining the LD50 (the dose lethal to 50% of the population) and the ED50 (the dose therapeutically effective in 50% of the population). The dose ratio between the toxic and therapeutic effects is the therapeutic index and it can be expressed as the ratio between LD50 and ED50. The data obtained from cell culture assays and animal studies can be used in formulating a range of dosage for use in human. The dosage of such compounds lies preferably within a range of circulating concentrations that include the ED50 with minimal toxicity. The dosage may vary within this range depending upon the dosage form employed and the route of administration utilized. Additional relative dosages, represented as a percent of maximal response or of maximum tolerated dose, are readily obtained via the protocols.

In some embodiments, the amount of a given formulation comprising compound 1, or a pharmaceutically acceptable salt thereof that corresponds to such an amount varies depending upon factors such as the molecular weight of a particular salt or form, disease condition and its severity, the identity (e.g., age, weight, sex) of the subject or host in need of treatment, but can nevertheless be determined according to the particular circumstances surrounding the case, including, e.g., the specific agent being administered, the liquid formulation type, the condition being treated, and the subject or host being treated.

In some embodiments, the amount of Compound 1, or a pharmaceutically acceptable salt thereof, as described herein is relative to the free-base equivalent of Compound 1.

In some embodiments, the amount of Compound 1, or a pharmaceutically acceptable salt thereof, administered is between 1 mg and 500 mg per day.

In some embodiments, the amount of Compound 1, or a pharmaceutically acceptable salt thereof, administered is between 1 mg and 400 mg per day.

In some embodiments, the amount of Compound 1, or a pharmaceutically acceptable salt thereof, administered is between 1 mg and 300 mg per day.

In some embodiments, the amount of Compound 1, or a pharmaceutically acceptable salt thereof, administered is between 1 mg and 200 mg per day.

In some embodiments, the amount of Compound 1, or a pharmaceutically acceptable salt thereof, administered is between 1 mg and 100 mg per day.

In some embodiments, the amount of Compound 1, or a pharmaceutically acceptable salt thereof, administered is between 100 mg and 500 mg per day.

In some embodiments, the amount of Compound 1, or a pharmaceutically acceptable salt thereof, administered is between 200 mg and 500 mg per day.

In some embodiments, the amount of Compound 1, or a pharmaceutically acceptable salt thereof, administered is between 300 mg and 500 mg per day.

In some embodiments, the amount of Compound 1, or a pharmaceutically acceptable salt thereof, administered is between 400 mg and 500 mg per day.

In some embodiments, Compound 2, or a pharmaceutically acceptable salt thereof, is administered orally.

In some embodiments, the amount of Compound 2, or a pharmaceutically acceptable salt thereof, as described herein is relative to the free-base equivalent of Compound 2.

In some embodiments, the amount of Compound 2, or a pharmaceutically acceptable salt thereof, administered is between 1 mg and 100 mg per day.

In some embodiments, the amount of Compound 2, or a pharmaceutically acceptable salt thereof, administered is between 1 mg and 80 mg per day.

In some embodiments, the amount of Compound 2, or a pharmaceutically acceptable salt thereof, administered is between 1 mg and 60 mg per day.

In some embodiments, the amount of Compound 2, or a pharmaceutically acceptable salt thereof, administered is between 1 mg and 40 mg per day.

In some embodiments, the amount of Compound 2, or a pharmaceutically acceptable salt thereof, administered is between 1 mg and 20 mg per day.

### Administration

Administration of Compound 1, or a pharmaceutically acceptable salt thereof, and combination partners described herein are at a dosage described herein or at other dose levels and compositions determined and contemplated by a medical practitioner. In certain therapeutic applications, Compound 1, or a pharmaceutically acceptable salt thereof, and combination partners described herein, are administered to a patient already suffering from a disease in an amount sufficient to cure the disease or at least partially arrest or ameliorate the symptoms. Amounts effective for this use depend on the age of the patient, severity of the disease, previous therapy, the patient's health status, weight, and response to the compositions, and the judgment of the treating physician. Therapeutically effective amounts are optionally determined by methods including, but not limited to, a dose escalation clinical trial.

In certain embodiments wherein the patient's condition does not improve, upon the doctor's discretion the administration of a composition described herein are administered chronically, that is, for an extended period of time, including throughout the duration of the patient's life in order to ameliorate or otherwise control or limit the symptoms of the patient's disease. In other embodiments, administration of a composition continues until complete or partial response of a disease.

In some embodiments, Compound 1, or a pharmaceutically acceptable salt thereof, is administered once daily. In some embodiments, Compound 1, or a pharmaceutically acceptable salt thereof, is administered twice daily. In some embodiments, Compound 1, or a pharmaceutically acceptable salt thereof, is administered three times daily.

In some embodiments, Compound 2, or a pharmaceutically acceptable salt thereof, is administered once daily. In some embodiments, Compound 2, or a pharmaceutically acceptable salt thereof, is administered twice daily. In some embodiments, Compound 2, or a pharmaceutically acceptable salt thereof, is administered three times daily.

In some embodiments, Compound 1, or a pharmaceutically acceptable salt thereof, and combination partners described herein, are administered once a day. In some embodiments, Compound 1, or a pharmaceutically acceptable salt thereof, and combination partners described herein are administered twice a day. In some embodiments, Compound 1, or a pharmaceutically acceptable salt thereof, and combination partners described herein are administered three times a day.

In some embodiments, Compound 2, or a pharmaceutically acceptable salt thereof, and combination partners described herein, are administered once a day. In some embodiments, Compound 2, or a pharmaceutically acceptable salt thereof, and combination partners described herein are administered twice a day. In some embodiments, Compound 2, or a pharmaceutically acceptable salt thereof, and combination partners described herein are administered three times a day.

In some embodiments, Compound 1, or a pharmaceutically acceptable salt thereof, and combination partners described herein are administered to a subject who is in a fasted state. A fasted state refers to a subject who has gone without food or fasted for a certain period of time. General fasting periods include at least 4 hours, at least 6 hours, at least 8 hours, at least 10 hours, at least 12 hours, at least 14 hours and at least 16 hours without food. In some embodiments, Compound 1, or a pharmaceutically acceptable salt thereof, is administered to a subject who is in a fasted state for at least 8 hours. In other embodiments, Compound 1, or a pharmaceutically acceptable salt thereof, and combination partners described herein, are administered to a subject who is in a fasted state for at least 10 hours. In yet other embodiments, Compound 1, or a pharmaceutically acceptable salt thereof, and combination partners described herein, are administered to a subject who is in a fasted state for at least 12 hours. In other embodiments, Compound 1, or a pharmaceutically acceptable salt thereof, and combination partners described herein, are administered to a subject who has fasted overnight.

In other embodiments, Compound 1, or a pharmaceutically acceptable salt thereof, and combination partners described herein, are administered to a subject who is in a fed state. A fed state refers to a subject who has taken food or has had a meal. In certain embodiments, a composition is administered to a subject in a fed state 5 minutes post-meal, 10 minutes post-meal, 15 minutes post-meal, 20 minutes post-meal, 30 minutes post-meal, 40 minutes post-meal, 50 minutes post-meal, 1 hour post-meal, or 2 hours post-meal. In certain instances, Compound 1, or a pharmaceutically acceptable salt thereof, is administered to a subject in a fed state 30 minutes post-meal. In other instances, Compound 1, or a pharmaceutically acceptable salt thereof, and combination partners described herein, are administered to a subject in a fed state 1 hour post-meal. In yet further embodiments, Compound 1, or a pharmaceutically acceptable salt thereof, is administered to a subject with food.

In some embodiments, Compound 2, or a pharmaceutically acceptable salt thereof, and combination partners described herein are administered to a subject who is in a fasted state. A fasted state refers to a subject who has gone without food or fasted for a certain period of time. General fasting periods include at least 4 hours, at least 6 hours, at least 8 hours, at least 10 hours, at least 12 hours, at least 14 hours and at least 16 hours without food. In some embodiments, Compound 2, or a pharmaceutically acceptable salt thereof, is administered to a subject who is in a fasted state for at least 8 hours. In other embodiments, Compound 2, or a pharmaceutically acceptable salt thereof, and combination partners described herein, are administered to a subject who is in a fasted state for at least 10 hours. In yet other embodiments, Compound 2, or a pharmaceutically acceptable salt thereof, and combination partners described herein, are administered to a subject who is in a fasted state for at least 12 hours. In other embodiments, Compound 2, or a pharmaceutically acceptable salt thereof, and combination partners described herein, are administered to a subject who has fasted overnight.

In other embodiments, Compound 2, or a pharmaceutically acceptable salt thereof, and combination partners described herein, are administered to a subject who is in a fed state. A fed state refers to a subject who has taken food or has had a meal. In certain embodiments, a composition is administered to a subject in a fed state 5 minutes post-meal, 10 minutes post-meal, 15 minutes post-meal, 20 minutes post-meal, 30 minutes post-meal, 40 minutes post-meal, 50 minutes post-meal, 1 hour post-meal, or 2 hours post-meal. In certain instances, Compound 2, or a pharmaceutically acceptable salt thereof, is administered to a subject in a fed state 30 minutes post-meal. In other instances, Compound 2, or a pharmaceutically acceptable salt thereof, and combination partners described herein, are administered to a subject in a fed state 1 hour post-meal. In yet further embodiments, Compound 2, or a pharmaceutically acceptable salt thereof, is administered to a subject with food.

The length of a treatment cycle depends on the treatment being given. In some embodiments, the length of a treatment cycle ranges from two to six weeks. In some embodiments, the length of a treatment cycle ranges from three to six weeks. In some embodiments, the length of a treatment cycle ranges from three to four weeks. In some embodiments, the length of a treatment cycle is three weeks (or 21 days). In some embodiments, the length of a treatment cycle is four weeks (28 days). In some embodiments, the length of a treatment cycle is five weeks (35 days). In some embodiments, the length of a treatment cycle is 56 days. In some embodiments, a treatment cycle lasts one, two, three, four, or five weeks. In some embodiments, a treatment cycle lasts three weeks. In some embodiments, a treatment cycle lasts four weeks. In some embodiments, a treatment cycle lasts five weeks. The number of treatment doses scheduled within each cycle also varies depending on the drugs being given.

### Example 1: Inhibition of MTAP-Deleted Kras-Mutant Cell Viability by Combination of Compound 2 with a PRMT5 Inhibitor

This Example illustrates that Compound 2 inhibits cell viability of MTAP-deleted KRAS-mutant cell lines with the combination of a PRMT5 inhibitor.

MTAP-delated KRAS-mutant cell lines, SU86.86 (ATCC, CRL-1837), Capan-1 (ATCC, HTB-79), HuP-T3 (DSMZ, ACC-259), LU99 (RCB, RCB1900), and PSN-1 (ATCC, CRL-3211) were cultured according to published protocols and maintained at 37° C. in 5% CO₂.

For combination assay, five days post-treatment, cell viability was assessed using either Cell CellTiter-Glo® Luminescent 3D Cell Viability assay (Promega #G9683) or CellTiter-Glo® Luminescent Cell Viability Assay (Promega #G7573). Cells were plated in a 96-well ULA plate (Corning #4520) for CellTiter-Glo® Luminescent 3D Cell Viability assay (Promega #G9683) or in a flat-bottom 96-well plate (Corning #3917) for CellTiter-Glo® Luminescent Cell Viability Assay (Promega #G7573) at the indicated cell density (Table 1). Cells were allowed to adhere overnight and treated the following day with a Tecan D300e Digital Dispenser (Tecan Group Ltd., Switzerland). The combination matrix (one compound serially diluted 3 (or 4) folds for 7 concentration points plus 0 nM and the other compound serially diluted 3 folds for 10 concentration points plus 0 nM) was applied for the assay. The combination effect was calculated based on HSA method and visualized using in-house software.

HSA (highest single agent) model was utilized for synergy score calculation (Berenbaum MC, Pharmacol Rev. 1989; 41:93-141). HSA is one of the reference models, which states that the expected combination effect is the maximum of the single drug responses at corresponding concentrations. Thus, HSA synergy score, S_{HSA}, is defined as:

$${S}_{HSA} = {E}_{A , B , … , N} - max ( {E}_{A} , {E}_{B} , … , {E}_{N} ) ,$$

where E_{A, B, . . . , N} is the combination effect between N drugs and E_{A}, E_{B}, . . . , E_{N} are the measured responses of the single drugs.

TABLE 1

| | | Catalog | | Cell |
|---|---|---|---|---|
| Cell line | Source | Nr. | Media | density |
| SU86.86 | ATCC | CRL-1837 | RPMI1640, 10% FBS, 1% Pen/Strep | 5K/well |
| Capan-1 | ATCC | HTB-79 | IMDM, 20% FBS, 1% Pen/Strep | 5K/well |
| HuP-T3 | DSMZ | ACC-259 | EMEM, 10% FBS, 1 mM non-essential amino | 1K/well |
| | | | acids, 1 mM sodium pyruvate, 1% Pen/Strep | |
| LU99 | RCB | RCB1900 | RPMI1640, 10% FBS, 1% Pen/Strep | 5K/well |
| PSN-1 | ATCC | CRL-3211 | RPMI1640, 10% FBS, 1% Pen/Strep | 5K/well |

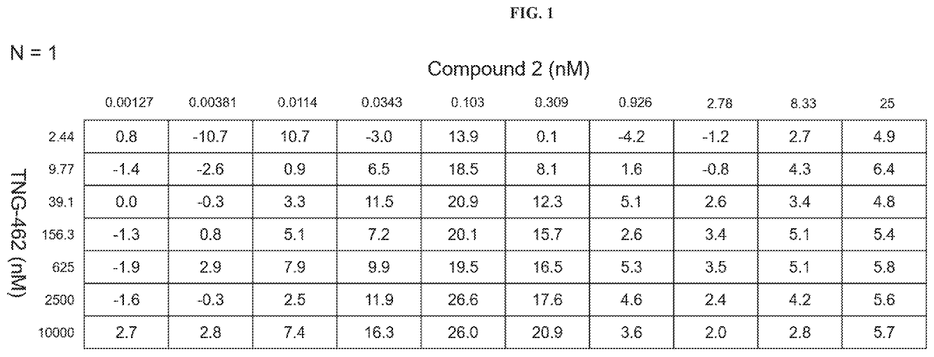

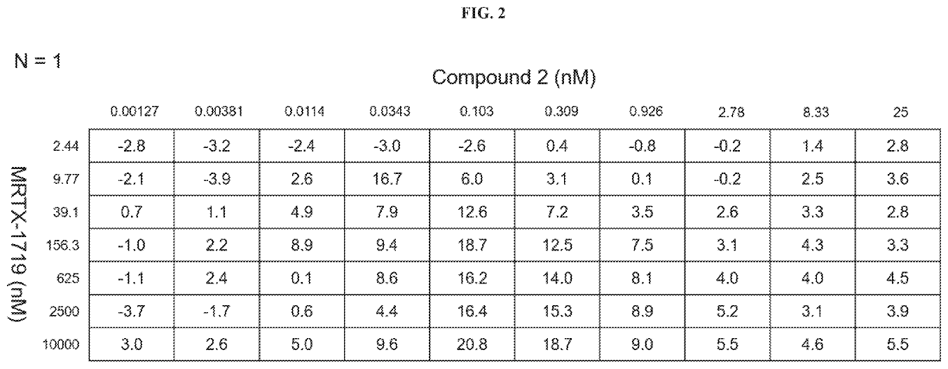

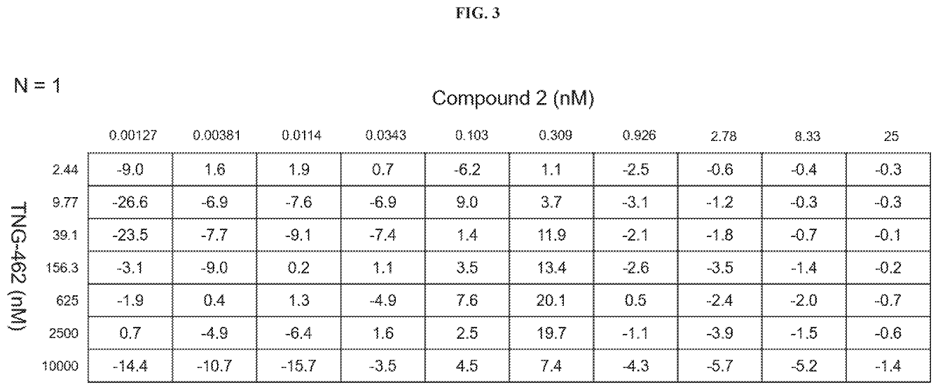

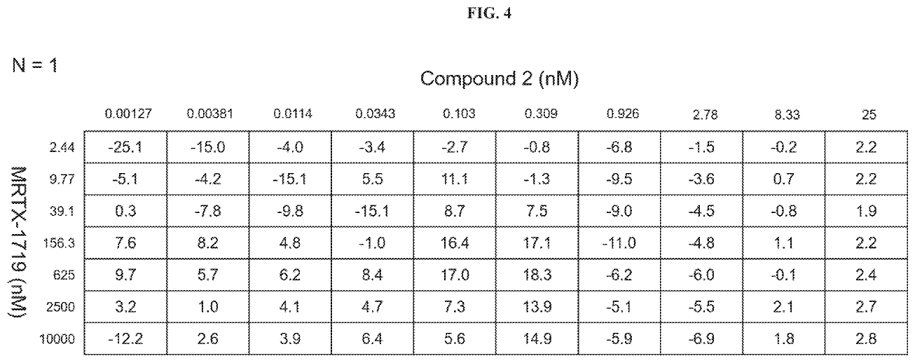

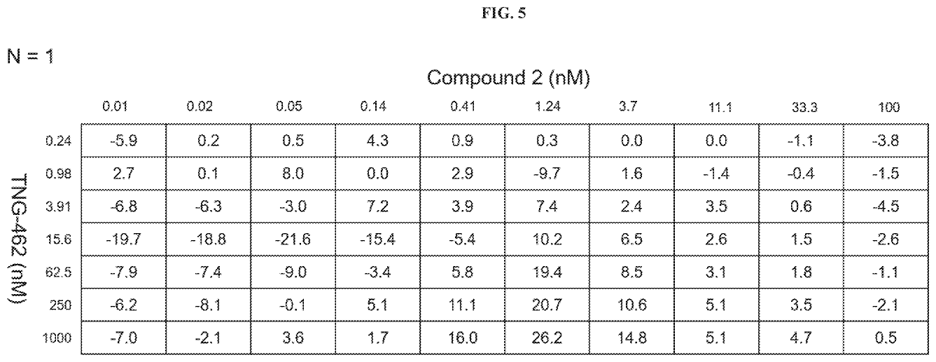

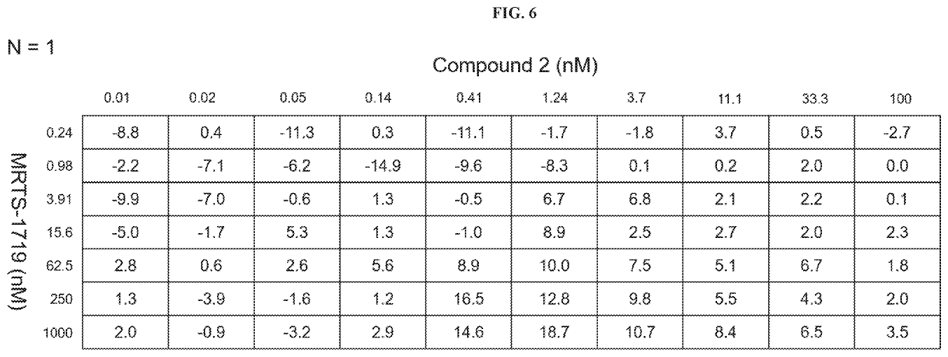

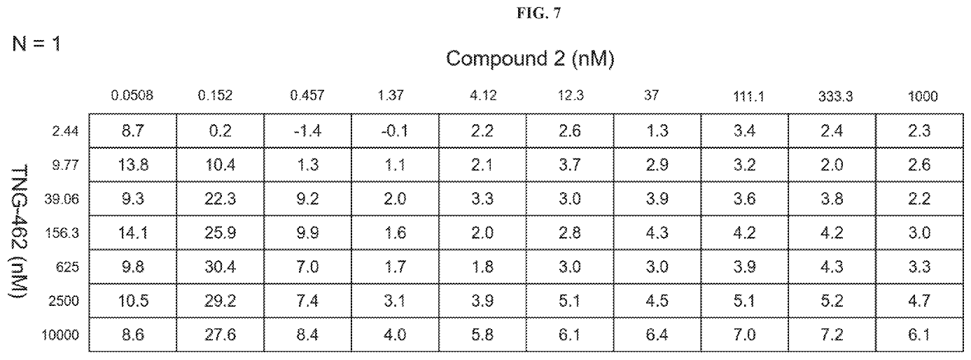

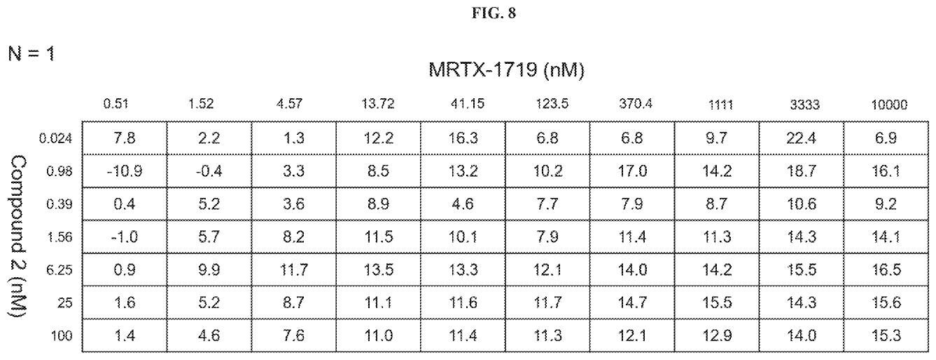

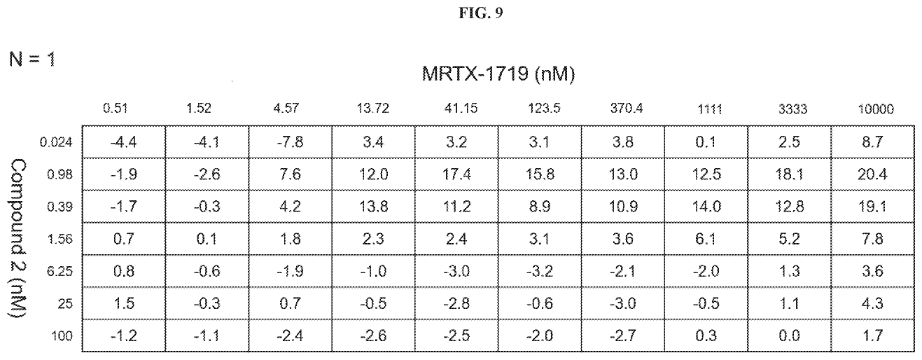

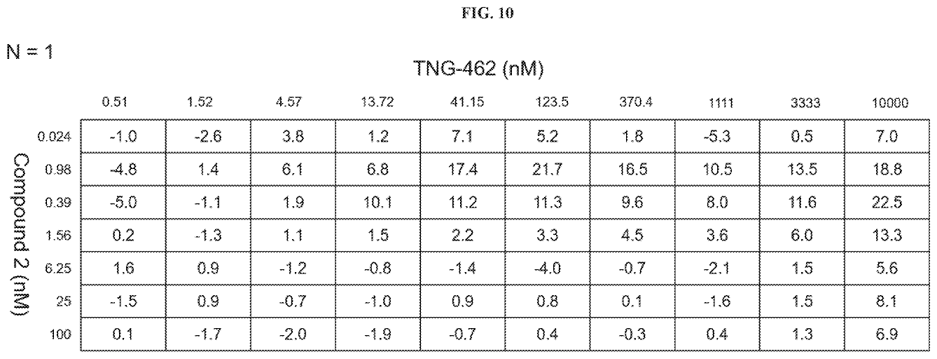

FIGS. 1-10 provide the HSA synergy and antagonism tables of Compound 2 and TNG-462 or MRTX-1719 as PRMT5 inhibitor. ‘+’ indicates synergy and ‘−’ indicates antagonism.

### Example 2: Inhibition of MTAP-Deleted Kras-Mutant Cell Viability by Combination of Compound 1 and Compound 2 with a PRMT5 Inhibitor

For a triple combination assay, five days post-treatment, cell viability is assessed using either Cell CellTiter-Glo® Luminescent 3D Cell Viability assay (Promega #G9683) or CellTiter-Glo® Luminescent Cell Viability Assay (Promega #G7573). Cells are plated in a 96-well ULA plate (Corning #4520) for CellTiter-Glo® Luminescent 3D Cell Viability assay (Promega #G9683) or in a flat-bottom 96-well plate (Corning #3917) for CellTiter-Glo® Luminescent Cell Viability Assay (Promega #G7573) at the indicated cell density (Table 1). Cells are allowed to adhere overnight and treated the following day with a Tecan D300e Digital Dispenser (Tecan Group Ltd., Switzerland). The combination matrix (the first compound for the x axis (Compound 1; starting concentration is 1000 nM) serially is diluted 3 (or 4) folds for 7 concentration points plus 0 nM, the second compound for the y axis (Compound 2; starting concentration is 25 nM) serially diluted 3 folds for 10 concentration points plus 0 nM, and the concentration of the third compound for the z axis (PRMT5i; serially diluted 4 folds starting from 1000 nM) is fixed) is applied for the assay. For the complete data set, the matrix is applied for 7 more plates with the fixed concentration (serially diluted 4 folds starting from 250 nM plus 0 nM) of the third compound (PRMT5i). The combination effect can be calculated with known calculation method like HSA and visualized using the in-house software.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

What is claimed is:

1. A method of treating cancer in a subject in need thereof, the method comprising:
administering to the subject in need thereof a therapeutically effective amount of:
(i) Compound 2: 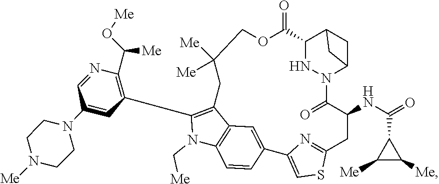
or a pharmaceutically acceptable salt thereof, and
(ii) TNG-462;
wherein the cancer is an MTAP-deleted cancer (MTAP^{del}).

2. The method of claim 1, wherein the cancer is a RAS-dependent cancer.

3. The method of claim 1, wherein the cancer comprises a RAS mutation.

4. The method of claim 1, wherein the RAS mutation is a KRAS, a NRAS, and/or a HRAS mutation.

5. The method of claim 4, wherein the RAS mutation is a mutation at the G12, G13, and/or Q61 position of the RAS protein.

6. The method of claim 5, wherein the RAS mutation is a G12C mutation of the RAS protein.

7. The method of claim 5, wherein the RAS mutation is a G12D mutation of the RAS protein.

8. The method of claim 5, wherein the RAS mutation is a G12R mutation of the RAS protein.

9. The method of claim 5, wherein the RAS mutation is a G12V mutation of the RAS protein.

10. The method of claim 1, wherein the cancer is a solid tumor.

11. The method of claim 1, wherein the cancer is breast cancer, gastrointestinal cancer, head and neck cancer, lung cancer, liver cancer, melanoma, ovarian cancer, pancreatic cancer, salivary gland tumor, thyroid cancer, or uterine cancer.

12. The method of claim 11, wherein the lung cancer is non-small cell lung cancer (NSCLC).

13. The method of claim 11, wherein the pancreatic cancer is pancreatic ductal adenocarcinoma (PDAC).

14. The method of claim 11, wherein the gastrointestinal cancer is anal cancer, appendiceal cancer, bile duct cancer, cholangiocarcinoma cancer, colon cancer, colorectal cancer (CRC), gallbladder cancer, rectal cancer, small intestine cancer, or stomach cancer (gastric cancer).

15. The method of claim 11, wherein the gastrointestinal cancer is colorectal cancer (CRC).

16. The method of claim 1, wherein the amount of Compound 2, or a pharmaceutically acceptable salt thereof, administered is between 1 mg and 100 mg per day.

17. The method of claim 1, wherein the amount of Compound 2, or a pharmaceutically acceptable salt thereof, administered is between 1 mg and 80 mg per day.

18. The method of claim 1, wherein the amount of Compound 2, or a pharmaceutically acceptable salt thereof, administered is between 1 mg and 60 mg per day.

19. The method of claim 1, wherein the amount of Compound 2, or a pharmaceutically acceptable salt thereof, administered is between 1 mg and 40 mg per day.

20. The method of claim 1, wherein the amount of Compound 2, or a pharmaceutically acceptable salt thereof, administered is between 1 mg and 20 mg per day.
